(19) United States
(12) Patent Application Publication
(10) Pub. No.: US 2023/0381239 A1
(43) Pub. Date: Nov. 30, 2023

(54) Use of Milk Fat Globule Membrane

(71) Applicant: Mead Johnson Nutrition Company, Evansville, IN (US)

(72) Inventors: Steven Shi-fong Wu, Evansville, IN (US); Colin David Rudolph, San Francisco, CA (US)

(21) Appl. No.: 18/198,753

(22) Filed: May 17, 2023

Related U.S. Application Data
(60) Provisional application No. 63/347,135, filed on May 31, 2022.

(51) Int. Cl. A61K 35/20; A61K 38/40; A61K 31/702; A61K 31/202; A61P 25/28; A23L 33/19; A23L 33/00; A23L 33/12; A23L 33/125

(52) U.S. Cl. CPC A61K 35/20; A61K 38/40; A61K 31/702; A61K 31/202; A61P 25/28; A23L 33/19; A23L 33/40; A23L 33/12; A23L 33/125

(57) ABSTRACT

The present application provides a synthetic nutritional composition comprising milk fat globule membrane (MFGM) in the range of about 10 μg/100 kcal to about 1500 mg/100 kcal for use in improving neurological development in a paediatric subject. The synthetic nutritional composition is administered to the paediatric subject for at least three months during the first year of life and said administration of the synthetic nutritional composition improves neurological development of the paediatric subject from at least two years of age.

### CROSS-REFERENCE TO RELATED APPLICATIONS

This application claims the benefit under 35 USC § 119(e), of U.S. Provisional Patent Application No. 63/347,135, filed 31 May 2022, the entire contents and substance of which are incorporated herein by reference in its entirety as if fully set forth below.

### FIELD OF THE INVENTION

The present application relates to a synthetic nutritional composition comprising milk fat globule membrane (MFGM) for use in improving neurological development in a paediatric subject, comprising the step of administering to the subject a synthetic nutritional composition comprising milk fat globule membrane (MFGM). More particularly, the present application relates to a synthetic nutritional composition comprising MFGM in the range of about 10 μg/100 kcal to about 1500 mg/100 kcal for use in improving neurological development in a paediatric subject, wherein the synthetic nutritional composition is administered to the paediatric subject for at least three months during the first year of life, and wherein said administration of the synthetic nutritional composition improves neurological development of the paediatric subject from at least two years of age.

### BACKGROUND

Human milk contains a number of components that contribute to the growth and development of the brain in infants. Observational studies of infant cognitive function have shown an advantage for breastfed infants over formula-fed infants, even after control for socioeconomic factors. Changes in infant formula to better match the dynamic features of breast milk have brought the composition, functionality, and health-based outcomes closer together for infants receiving human milk or infant formula.

Synthetic nutritional compositions for human consumption based on bovine dairy products are, typically, made using skimmed milk powder as the predominant dairy component. Skimmed milk powder contains small quantities of bioactive components such as phospholipids, sphingolipids and gangliosides. Some of these bioactive components have been shown to have a profound and lasting impact on human brain function, particularly on the brain function of infants whilst they are developing. Whilst skimmed milk powder contains these beneficial bioactive components, they are only present in small quantities which would not be sufficient to provide the desired benefits to human brain function.

A number of these bioactive components are present within milk fat globule membrane (MFGM). MFGM is a naturally occurring bioactive membrane structure that surrounds the fat droplets in human milk and other mammalian milk e.g. cow's milk. MFGM is comprised of a trilayer lipid structure that comprises a complex mixture of phospholipids (such as phosphatidylethanolamine, phosphatidylcholine, phosphatidylserine, and phosphatidylinositol), glycolipids, glycosphingolipids (such as sphingomyelin and gangliosides), other polar lipids, proteins, glycoproteins (such as xanthine dehydrogenase, xanthine oxidase, lactadherin, fatty acid binding proteins (FABPs), mucin-1, butyrophilins, adipophilin, and cluster of differentiation 36 (CD36)), triglycerides, cholesterol, 7-dehydrocholesterol, enzymes, and other components.

There are reports of benefits in neurological development in paediatric subjects of 12-18 months of age through supplementation of infant formula with MFGM during the first year of life.

Timby et al., Am J Clin Nutr 2014; 99:860-8 reports a study wherein infants were fed an experimental formula with added MFGM (EF) or a standard formula (SF) until six months of age. At 12 months of age, the cognitive score on testing with the Bayley Scales of Infant and Toddler Development, Third edition (“Bayley-III”), was higher in the EF group than in the SF group and not significantly different from that in the reference breast-fed group of infants.

Li et al., J Pediatr 2019, 215, 24-31 reports a study wherein infants were fed an infant formula with added MFGM and lactoferrin (MFGM+Lf) or a standard cows' milk-based infant formula (control) until six months of age. The primary outcome was the Bayley-III cognitive score at day 365 (i.e. 12 months of age). At 12 months of age, the cognitive score on testing with Bayley-III was higher in the MFGM+Lf group than in the control group. However, no group differences were detected in any Bayley-III domain (cognitive, language and motor) at day 545 of life (i.e. 18 months of age), although scores of some subcategories of the MacArthur-Bates Communicative Development Inventories were higher (p<0.05) in the MFGM+Lf group. The MFGM+Lf group achieved higher scores compared to the control group at 18 months of age in sentence complexity (using words in longer and more grammatically correct combinations) and two categorical items: absent owners (naming an absent person to whom a visible object belongs) and Chinese classifiers (grammatical marker specific to Chinese language).

It is desirable to provide synthetic nutritional compositions for administration to paediatric subjects of up to 12 months of age that provide neurological benefits which persist beyond 12 months or even 18 months of age.

### SUMMARY OF INVENTION

According to a first aspect of the invention there is provided a synthetic nutritional composition comprising milk fat globule membrane (MFGM) in the range of about 10 μg/100 kcal to about 1500 mg/100 kcal for use in improving neurological development in a paediatric subject,
wherein the synthetic nutritional composition is administered to the paediatric subject for at least three months during the first year of life,
and wherein said administration of the synthetic nutritional composition improves neurological development of the paediatric subject from at least two years of age.

Preferably, the improvement in neurological development is an improvement in cognitive development.

Preferably, the improvement in cognitive development is selected from at least one of an improvement in the development of memory, verbal comprehension, visual spatial ability, fluid reasoning, mental processing speed, rule learning, executive function and language acquisition.

Preferably, the administration of the synthetic nutritional composition takes place for at least six months during the first year of life.

Preferably, the administration of the synthetic nutritional composition takes place from three months of age to six months of age.

Preferably, the improvement in the neurological development of the paediatric subject from at least two years of age persists until at least five years of age.

Preferably, the synthetic nutritional composition comprises MFGM in the range of about 15 μg/100 kcal to about 1000 mg/100 kcal, preferably about 100 μg/100 kcal to about 500 mg/100 kcal.

Preferably, the synthetic nutritional composition comprises phospholipids in the range of about 6 mg/100 kcal to about 300 mg/100 kcal, preferably about 12 mg/100 kcal to about 150 mg/100 kcal, more preferably about 18 mg/100 kcal to about 85 mg/100 kcal.

Preferably, the synthetic nutritional composition comprises sphingomyelin in a range of about 1 mg/100 kcal to about 30 mg/100 kcal, preferably about 3.5 mg/100 kcal to about 24 mg/100 kcal, more preferably about 6 mg/100 kcal to about 21 mg/100 kcal.

Preferably, the synthetic nutritional composition comprises gangliosides in a range of about 0.25 mg/100 kcal to about 6 mg/100 kcal, preferably about 0.7 mg/100 kcal to about mg/100 kcal, more preferably about 1.1 mg/100 kcal to about 4.5 mg/100 kcal.

Preferably, the MFGM is provided by a whey protein concentrate. Preferably, the synthetic nutritional composition comprises the whey protein concentrate in a range of about 6.5 to about 12.0 wt % on a dry weight basis, preferably about 7.9 wt % to about 10.3 wt %.

Preferably, the MFGM is provided by an enriched milk product. Preferably, the synthetic nutritional composition comprises the enriched milk product in the range of about g/100 kcal to about 1.5 g/100 kcal, preferably about 0.3 g/100 kcal to about 1.4 g/100 kcal, more preferably about 0.4 g/100 kcal to about 1 g/100 kcal.

Preferably, the MFGM is provided by buttermilk. Preferably, the synthetic nutritional composition comprises buttermilk in the range of about 0.06 g/100 kcal to about 10.5 g/100 kcal, preferably about 0.3 g/100 kcal to about 8.5 g/100 kcal, more preferably about 0.4 g/100 kcal to about 7 g/100 kcal.

Preferably, the synthetic nutritional composition further comprises lactoferrin. Preferably, the synthetic nutritional composition comprises lactoferrin in the range of about 5 mg/100 kcal to about 300 mg/100 kcal, preferably about 60 mg to about 150 mg/100 kcal, more preferably about 60 mg/100 kcal to about 100 mg/100 kcal.

Preferably, the synthetic nutritional composition further comprises polydextrose (PDX) and/or galactooligosaccharides (GOS). Preferably, the synthetic nutritional composition comprises PDX in the range of about 0.015 g/100 kcal to about 1.5 g/100 kcal, preferably about 0.05 g/100 kcal to about 1.5 g/100 kcal, more preferably about 0.2 g/100 kcal to about 0.6 g/100 kcal. Preferably, the synthetic nutritional composition comprises GOS in the range of about 0.015 g/100 kcal to about 1.0 g/100 kcal, preferably about 0.2 g/100 kcal to about 0.5 g/100 kcal.

Preferably, the synthetic nutritional composition further comprises at least one human milk oligosaccharide (HMO) selected from the group consisting of 2′-fucosyllactose (2FL), 3′-fucosyllactose (3FL), lacto-N-tetraose (LNT), lacto-N-neotetraose (LNnT), lacto-N-fucopentaose I (LNFP-I), 3′-sialyllactose (3SL), 6′-sialyllactose (6SL), or any combination thereof. Preferably, the synthetic nutritional composition comprises the HMO in the range of about 0.01 g/100 kcal to about 2.0 g/100 kcal, preferably about 0.01 g/100 kcal to about 1.5 g/100 kcal.

Preferably, the synthetic nutritional composition further comprises a source of long-chain polyunsaturated fatty acids (LCPUFAs) comprising docosahexaenoic acid (DHA), arachidonic acid (ARA), or a combination thereof. Preferably, the synthetic nutritional composition comprises the LCPUFA in the range of about 5 mg/100 kcal to about 100 mg/100 kcal, preferably about 10 mg/100 kcal to about 50 mg/100 kcal.

According to an aspect related to the first aspect of the invention, there is provided a method for improving neurological development in a paediatric subject, the method comprising the step of administering a synthetic nutritional composition comprising milk fat globule membrane (MFGM) in the range of about 10 μg/100 kcal to about 1500 mg/100 kcal, wherein the synthetic nutritional composition is administered to the paediatric subject for at least three months during the first year of life,
and wherein said administration of the synthetic nutritional composition improves neurological development of the paediatric subject from at least two years of age.

According to an aspect related to the first aspect of the invention, there is provided the use of a synthetic nutritional composition comprising milk fat globule membrane (MFGM) in the range of about 10 μg/100 kcal to about 1500 mg/100 kcal in the manufacture of a medicament for improving neurological development in a paediatric subject,
wherein the synthetic nutritional composition is administered to the paediatric subject for at least three months during the first year of life,
and wherein said administration of the synthetic nutritional composition improves neurological development of the paediatric subject from at least two years of age.

### BRIEF DESCRIPTION OF THE FIGURE

FIG. 1A-10 show graphs comparing neurophysiologic syllable sound perception in paediatric subjects of 2 years of age. Results are presented for infants who received infant formula up to 12 months of age (SF and EF) and a reference group of infants exclusively receiving human milk (HM).

FIG. 1A illustrates the overall P1 Amplitude (native familiar stimuli, native unfamiliar stimuli and foreign stimuli combined) by group. FIG. 1B illustrates the overall P1 Latency by group.

FIG. 10 illustrates the P1 Latency in response to native unfamiliar stimuli (NUS) by group.

### DEFINITIONS

“Milk” means a substance that has been drawn or extracted from the mammary gland of a mammal.

“Milk-based composition” means a composition comprising any mammalian milk-derived or mammalian milk-based product known in the art. For example, a “milk-based composition” may comprise bovine casein, bovine whey, bovine lactose, bovine milk fat globule membrane (MFGM), bovine milk fat, or any combination thereof.

“Enriched milk product” generally refers to a milk ingredient that has been enriched with MFGM and/or certain MFGM components, such as proteins and lipids found in the MFGM and possessing a fat content of between 14 to 20 wt %. By way of example, Lacprodan MFGM-10 available from Arla Foods Ingredients is a form of an enriched milk product.

“Lactoferrin” refers to lactoferrin that is produced by or obtained from a source other than human breast milk.

“Paediatric subject” means a human under 18 years of age. The term “paediatric subject” may refer to preterm infants, full-term infants, and/or children, as described below. A paediatric subject may be a human subject that is between birth and 8 years old. In another aspect, “paediatric subject” refers to a human subject between 1 and 6 years of age. Alternatively, “paediatric subject” refers to a human subject between 6 and 13 years of age, or between 6 and 12 years of age.

“Infant” means a human subject ranging in age from birth to not more than one year and includes infants from 0 to 12 months corrected age. The phrase “corrected age” means an infant's chronological age minus the amount of time that the infant was born premature. Therefore, the corrected age is the age of the infant if it had been carried to full term. The term infant includes full-term infants, preterm infants, low birth weight infants, very low birth weight infants, and extremely low birth weight infants. “Preterm” means an infant born before the end of the 37^{th} week of gestation. “Full-term” means an infant born after the end of the 37^{th} week of gestation. “Low birth weight infant” means an infant born weighing less than 2500 grams (approximately 5 lbs, 8 ounces). “Very low birth weight infant” means an infant born weighing less than 1500 grams (approximately 3 lbs, 4 ounces). “Extremely low birth weight infant” means an infant born weighing less than 1000 grams (approximately 2 lbs, 3 ounces).

“Child” means a human subject ranging from 12 months to 13 years of age. A child may be a subject between the ages of 1 and 12 years old. In another aspect, the terms “children” or “child” may refer to subjects that are between 1 and about 6 years old. Alternatively, the terms “children” or “child” may refer to subjects that are between about 7 and about 12 years old. The term “young child” means a human subject ranging from 1 year to 3 years of age.

The term “synthetic” when applied to a composition, a nutritional composition, or a mixture means a composition, nutritional composition, or mixture obtained by biological and/or chemical means, which can be chemically identical to the mixture naturally occurring in mammalian milks. A composition, nutritional composition, or mixture is said to be “synthetic” if at least one of its components is obtained by biological (e.g. enzymatic) and/or chemical means.

“Nutritional composition” means a substance or composition that satisfies at least a portion of a subject's nutrient requirements. “Nutritional composition(s)” may refer to liquids, powders, solutions, gels, pastes, solids, concentrates, suspensions, ready-to-use forms of enteral formulas, oral formulas, formulas for infants, follow-up formulas, formulas for paediatric subjects, formulas for children, and/or young child milks.

“Infant formula” means a composition that satisfies at least a portion of the nutrient requirements of an infant and is, ideally, capable of providing a sole source of nutrition to the infant.

“Follow-up formula”, also referred to as a “follow-on formula” means a composition that satisfies at least a portion of the nutrient requirements of an infant from the 6^{th} month onwards, and for young children from 1 to 3 years of age.

“Liquid concentrate”, means a liquid that needs to be diluted before it is ready to be administered to, or consumed by, the subject.

The term “enteral” means deliverable through or within the gastrointestinal, or digestive, tract. “Enteral administration” includes oral feeding, intragastric feeding, transpyloric administration, or any other administration into the digestive tract. “Administration” is broader than “enteral administration” and includes parenteral administration or any other route of administration by which a substance is taken into a subject's body.

The “gut” of a subject may also be referred to as the gastrointestinal system, the gastrointestinal tract, the digestive system, and/or the digestive tract, of a subject.

The term “substantially free” means containing less than a functional amount of the specified component, typically less than 0.1% by weight, and includes 0% by weight of the specified ingredient.

As applied to nutrients, the term “essential” refers to any nutrient that cannot be synthesised by the body in amounts sufficient for normal growth, so it must be supplied by the diet. The term “conditionally essential” as applied to nutrients means that the nutrient must be supplied by the diet when adequate amounts of the precursor compound is unavailable to the body for endogenous synthesis to occur.

“Prebiotic” means a non-digestible food ingredient that beneficially affects the host by selectively stimulating the growth and/or activity of one or a limited number of bacteria in the digestive tract, which can improve the health of the host. Prebiotics exert health benefits, which may include, but are not limited to: selective stimulation of the growth and/or activity of one or a limited number of beneficial gut bacteria; stimulation of the growth and/or activity of ingested probiotic microorganisms; selective reduction in gut pathogens; and, favourable influence on gut short chain fatty acid profile. The prebiotic of the composition may be naturally-occurring, synthetic, or developed through the genetic manipulation of organisms and/or plants, whether such new source is now known or developed later.

The term “human milk oligosaccharides” or “HMOs” refers generally to a number of complex carbohydrates found in human breast milk.

“N-acetylated oligosaccharide(s)” means both “N-acetyl-lactosamine” and “oligosaccharide(s) containing N-acetyl-lactosamine”. They are neutral oligosaccharides having an N-acetyl-lactosamine residue. Suitable examples are LNT (lacto-N-tetraose) and LNnT (lacto-N-neotetraose).

The term “sialic acid” refers to a family of derivatives of neuraminic acid. N-acetylneuraminic acid (Neu5Ac) and N-glycolylneuraminic acid (Neu5Gc) are among the most abundant, naturally-found forms of sialic acid, especially Neu5Ac in human and cow's milk.

“Probiotic” means microorganisms, such as bacteria or yeast, which have been shown to exert a beneficial effect on the health of a host subject. Probiotics can usually be classified as ‘viable’ or ‘non-viable’. The term ‘viable probiotics’ refers to living microorganisms, with the amount of a viable probiotic being detailed in colony-forming units (CFU). Probiotics that have been heat-killed, or otherwise inactivated, are termed ‘non-viable probiotics’ i.e. non-living microorganisms. Non-viable probiotics may still retain the ability to favourably influence the health of the host even though they may have been heat-killed or otherwise inactivated.

The term “degree of hydrolysis” refers to the extent to which peptide bonds, within a protein, are broken by a hydrolysis method. The degree of protein hydrolysis for the purposes of characterising the hydrolysed protein component of the composition is easily determined by one of ordinary skill in the formulation arts, by quantifying the amino nitrogen to total nitrogen ratio (AN/TN) of the protein component of the selected composition. The amino nitrogen component is quantified by USP titration methods for determining amino nitrogen content, with the total nitrogen component being determined by the Kjeldahl method. These methods are well-known to one of ordinary skill in the analytical chemistry art.

The term “partially hydrolysed”, in terms of the present disclosure, means having a degree of hydrolysis which is greater than 0% but less than about 40%.

The term “extensively hydrolysed”, in terms of the present disclosure, means having a degree of hydrolysis which is greater than or equal to about 40%.

“Reconstituted solution”, in terms of the present disclosure, means the solution prepared when a diluent (e.g. water, saline, etc.) is added to an ingredient (e.g. a powder, a solution, a gel, a suspension, a paste, a solid, a liquid, a liquid concentrate, etc.).

“Executive function” means the ability to recognise, evaluate, and make a choice among a variety of alternative options and strategies. The term encompasses goal-directed behaviour, planning and/or cognitive flexibility.

All percentages, parts, and ratios as used herein are detailed by weight of the total composition, unless otherwise specified. All amounts specified as administered “per day” may be delivered in a single unit dose, in a single serving, or in two or more doses or servings administered over the course of a 24 hour period.

All references to singular characteristics or limitations in the present disclosure shall include the corresponding plural characteristic or limitation, and vice versa, unless otherwise specified or clearly implied to the contrary, by the context in which the reference is made.

All combinations of method or process steps disclosed herein can be performed in any order, unless otherwise specified or clearly implied to the contrary, by the context in which the referenced combination is made.

The compositions of the present disclosure can comprise, consist of, or consist essentially of any of the components described herein, as well as including any additional useful component.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

### DETAILED DESCRIPTION

The inventors have surprisingly found that administration of a synthetic nutritional composition comprising MFGM for at least three months during the first year of life improves neurological development in the paediatric subject later on in childhood, i.e. when the paediatric subject is at least two years of age.

As discussed above, it has previously been shown in Timby et al. 2014 that supplementation with MFGM in the first year of life has effects at 12 months of age and Li et al. 2019 shows that the addition of MFGM and lactoferrin to synthetic nutritional compositions, which are administered in the first year of life, has effects at 12 months of age and limited effects at 18 months of age.

As detailed in Example 1, the inventors conducted a follow-up study to the study detailed in Li et al. 2019 on participants aged between 5.5 and 6 years of age who completed study feeding during the first year of life (i.e. through day 365 of age). The inventors surprisingly found that feeding infants formula with added MFGM and lactoferrin through 12 months of age led to improved neurological development (relative to the control group) later in life between 5.5 and 6 years of age. The results suggest that the addition of MFGM and lactoferrin to infant formula has persistent effects in supporting neurological development during childhood.

Although the test formula in Example 1 included both MFGM and lactoferrin, there is clinical evidence that MFGM and components thereof support the development of language and cognitive domains (Li et al. (2019)).

A significant body of preclinical data have demonstrated that MFGM, or components thereof, including sphingomyelin, gangliosides, phosphatidylserine and phosphatidylcholine, play roles in neural development and function, gastrointestinal immune defence and gut health. Glycosylated proteins (mucin-1, mucin-15, butyrophilin, and lactadherin) and glycosylated sphingolipids from MFGM may promote the development of healthy infant gut microbiota by favouring beneficial Bifidobacterium species (Bourlieu et al., Current Opinion in Clinical Nutrition and Metabolic Care: March 2015, 18(2), 118-127).

Clinical reports also suggest that MFGM and components thereof may affect brain function directly. For example, a nutritional intervention via administration of sphingomyelin-fortified milk has a positive association with the neurodevelopment of low-birth-weight-infants (Tanaka et al. (2013), Brain & Development, 35(1), 45-52).

Therefore, it is hypothesised that similar neurological benefits to those observed in Example 1 would also be observed in the absence of lactoferrin.

This is supported by the results for the clinical study detailed in Example 2. As discussed in Example 2, the inventors found that infants who received infant formula with added MFGM during the first year of life showed a lower event-related potential (ERP) amplitude at two years of age, compared to both infants who received a standard formula and the breastfed infant control group. The lower ERP potential amplitudes observed in the MFGM group may reflect a higher degree of maturation of neural circuits. The inventors also found that the MFGM group at two years of age showed a lower ERP latency for a subset of stimuli (native unfamiliar stimuli), which may suggest improved myelination.

Therefore, the administration of synthetic nutritional compositions with added MFGM during the first year of life, on the basis of improved neural maturation and myelination, may have a higher likelihood of achieving improved neurodevelopmental outcomes at older ages, as illustrated by the results presented in Example 2.

The finding that the effects of MFGM, optionally in the presence of lactoferrin, persist later in childhood was unexpected based on the Timby et al. 2014 and Li et al. 2019 studies. The Timby et al. 2014 study did not measure neurological development beyond 12 months of age. As noted by the authors of Li et al. (2019), the observation that few group differences in neurological outcomes were detected at 18 months could indicate that the control group caught up developmentally and the earlier advantages observed in the MFGM+Lf group were not sustained. Whilst differences in certain language subcategories were observed at day 545, notably no differences were detected at day 365. The authors postulated that the differences in Bayley-III domains at day 365 and certain language subcategories at day 545 may be indicative of a developmental cascade (Masten A S, Cicchetti D. Developmental cascades. Dev Psychopathol. 2010; 22:491-5).

To the best of the inventors' knowledge, the discovery that the addition of MFGM to synthetic nutritional compositions, which are administered in the first year of life, enhances neurological development in paediatric subjects from two years of age has not previously been shown and certainly could not be predicted by the results of Timby et al. 2014 or Li et al. 2019.

The present invention therefore provides a synthetic nutritional composition comprising milk fat globule membrane (MFGM) in the range of about 10 μg/100 kcal to about 1500 mg/100 kcal for use in improving neurological development in a paediatric subject, wherein the synthetic nutritional composition is administered to the paediatric subject for at least three months during the first year of life, and wherein said administration of the synthetic nutritional composition improves neurological development of the paediatric subject from at least two years of age.

The paediatric subject may be a neurologically healthy paediatric subject. For example, the administration of the synthetic nutritional composition comprising MFGM during the first year of life as defined above may promote the healthy establishment of cognitive function in the paediatric subject from at least two years of age.

The paediatric subject may instead be of compromised neurological health. For example, the improvement in neurological development according to the invention may be the repair of, or reduction in the severity of cognitive function impairment in the paediatric subject. The administration of the synthetic nutritional composition comprising MFGM during the first year of life may result in the treatment of disorders associated with the delayed establishment of cognitive function or cognitive function impairment in a paediatric subject from at least two years of age. Non-limiting examples of disorders include delayed and/or impaired learning ability, loss of, or poor development of executive functions, memory impairment and a delay in language development.

The improvement in neurological development in a paediatric subject is relative to the neurological development observed at the same age when a standard infant formula is administered, i.e. an infant formula which is not supplemented with MFGM.

The improvement in neurological development may be an improvement measured in a clinical setting following consumption of the composition in accordance with the present invention.

As explained above, the use of MFGM may result in a higher degree of maturation of neural circuits and/or improved myelination. This would translate into a fundamental benefit for brain development and normal brain function.

The improvement in neurological development may be an improvement in cognitive development.

The improvement in cognitive development may be an improvement measured by at least one standardised clinical neuropsychological test. Non-limiting examples include the Bayley Scales of Infant Development, 3rd or 4th editions (“Bayley-III” and “Bayley-IV”).

The improvement in cognitive development may be selected from at least one of an improvement in the development of memory, verbal comprehension, visual spatial ability, fluid reasoning, mental processing speed, rule learning, executive function, language acquisition and summary or global intelligence quotient (IQ) score. Preferably, the improvement in cognitive development is selected from at least one of an improvement in the development of memory, verbal comprehension, visual spatial ability, fluid reasoning, mental processing speed, rule learning, executive function and language acquisition.

The improvement in the development of memory, verbal comprehension, visual spatial ability, fluid reasoning and/or mental processing speed may be an improvement measured by at least one standardised clinical neuropsychological test. A non-limiting example of a suitable neuropsychological test is the Wechsler Preschool & Primary Scale of Intelligence, Fourth Edition (WPPSI-IV).

Executive function is the ability to coordinate and integrate cognitive-perceptual processes in relation to time and space, determining how well a subject can recognise, evaluate and make a choice among a variety of alternative options and strategies. Skills that comprise executive function include attention, working memory, inhibitory control (e.g. rule learning) and cognitive flexibility.

The improvement in executive function may be an improvement measured by at least one standardised clinical neuropsychological test. Non-limiting examples of suitable neuropsychological tests are:
Stroop Task Test—demonstrates rule learning and inhibitory control
Dimensional Change Card Sort (DCCS) task—demonstrates rule learning and cognitive flexibility.

The improvement in language acquisition may be an improvement measured by at least one standard clinical neuropsychological test. Non-limiting examples include Bayley-III, Bayley-IV or auditory event-related potential measurements (Choudhury N, Benasich A A. Clin Neurophysiol: Official J Int Fed Clin Neurophysiol. 2011; 122(2):320-38; Riva V et al. Cereb Cortex. 2017; 28(6):2100-8.). In Example 2, auditory event-related potential measurements were carried out with stimuli presented in a mismatch negativity (MMN) paradigm.

The synthetic nutritional composition of the present invention is administered to an infant for at least three months during the first year of life. The administration can start from birth or some days/weeks/months later. The administration can be continuous, e.g. daily administration, or not. It may be administered throughout this time period, or during only a part thereof, for example after the first month of life of the infant such as from 0 to 6 months, 1 to 6 months, 2 to 6 months, 3 to 6 months or from 1 to 12 months, 2 to 12 months, 3 to 12 months, 4 to 12 months, 5 to 12 months, 6 to 12 months, 7 to 12 months, 8 to 12 months or 9 to 12 months of age.

The synthetic nutritional composition may be administered from three months of age to six months of age. For the avoidance of doubt, the administration may start prior to and/or continue beyond this specific age range.

The synthetic nutritional composition may be a single composition according to the present invention. Alternatively, different compositions according to the present invention may be administered that are intended for different age stages. For example, a stage 1 composition could be administered up to six months (or 180 days) of age and then a stage 2 composition could be administered from 6 months of age and up to 1 year of age.

The administration of the synthetic nutritional composition takes place for at least three months during the first year of life, or for a longer total time period such as at least four months, at least five months, at least six months, at least 7 months, at least 8 months, at least 9 months, at least 10 months or at least 11 months. Preferably, the administration of the synthetic nutritional composition takes place for at least six months during the first year of life.

In addition to the administration during the first year of life, the administration of the synthetic nutritional composition according to the present invention may continue beyond the first year of life. For example, in addition to the administration during the first year of life, the synthetic nutritional composition may be administered as a young child milk having a composition according to the present invention from 12 months (i.e. one year of age) to 18 months of age, or from 12 months to 24 months of age (i.e. two years of age), or from 12 months to three years of age. Again, the young child milk composition may be the same as the composition administered up to one year of age, or it may be different to the composition administered up to one year of age.

As mentioned, the administration of the synthetic nutritional composition during the first year of life improves neurological development in the paediatric subject from at least two years of age. Preferably, the improvement in the neurological development of the paediatric subject from at least two years of age persists until at least five years of age. The improvement may persist from 2 years of age until later on in childhood, such as up to 5 years, 6 years, 7 years, 8 years, 9 years, 10 years, 11 years, 12 years, or 13 years of age. Alternatively, the improvement may occur from 3 years of age and up to 13 years, up to 12 years, up to 11 years, up to 10 years, up to 9 years, up to 8 years, up to 7 years, or up to 6 years of age. Alternatively, the improvement may occur from 4 years of age and up to 13 years, up to 12 years, up to 11 years, up to 10 years, up to 9 years, up to 8 years, up to 7 years, or up to 6 years of age. Alternatively, the improvement may occur from 5 years of age and up to 13 years, up to 12 years, up to 11 years, up to 10 years, up to 9 years, up to 8 years, up to 7 years, or up to 6 years of age.

As mentioned, the synthetic nutritional composition comprises milk fat globule membrane (MFGM). MFGM is a naturally occurring bioactive membrane structure that surrounds the fat droplets in human milk and other mammalian milk e.g. cow's milk. MFGM is comprised of a trilayer lipid structure that comprises a complex mixture of phospholipids (such as phosphatidylethanolamine, phosphatidylcholine, phosphatidylserine, and phosphatidylinositol), glycolipids, glycosphingolipids (such as sphingomyelin and gangliosides), other polar lipids, proteins, glycoproteins (such as xanthine dehydrogenase, xanthine oxidase, lactadherin, fatty acid binding proteins (FABPs), mucin-1, butyrophilins, adipophilin, and cluster of differentiation 36 (CD36)), triglycerides, cholesterol, 7-dehydrocholesterol, enzymes, and other components.

The MFGM of any aspect detailed below may be provided by may be provided by a whey protein concentrate, an enriched milk product, buttermilk, or any combination thereof.

The whey protein concentrate, enriched milk product, and/or buttermilk may be derived from non-human milk sources, such as bovine whole milk, bovine cream, porcine milk, equine milk, buffalo milk, goat milk, murine milk, camel milk, or any combination thereof.

The composition comprises MFGM in the range of about 10 μg/100 kcal to about 1500 mg/100 kcal. Preferably, the composition comprises MFGM in the range of about 15 μg/100 kcal to about 1000 mg/100 kcal. More preferably, the composition comprises MFGM in the range of about 100 μg/100 kcal to about 500 mg/100 kcal.

When the composition comprises a reconstituted solution, the reconstituted solution may comprise MFGM in the range of about 0.5 mg/mL to about 1.5 mg/mL. Preferably, the reconstituted solution comprises MFGM in the range of about 0.6 mg/mL to about 1.3 mg/mL. Alternatively, the reconstituted solution may comprise MFGM in the range of about 0.1 grams per litre (g/L) to about 6 g/L. Preferably, the reconstituted solution comprises MFGM in the range of about 0.6 g/L to about 5 g/L.

The MFGM may be provided by a whey protein concentrate (WPC). The whey protein concentrate may be provided in any suitable form for making a synthetic nutritional composition, preferably the whey protein concentrate is provided in powdered form or provided in a liquid form. The whey protein content may have a milk fat content of between 6.5 to 10.0 wt %.

The synthetic nutritional composition for use according to the present invention preferably has a whey protein to casein protein ratio of between 60:40 to 100:0 on a dry weight basis; more preferably a whey protein to casein protein ratio of 60:40 to 80:20; more preferably a whey protein to casein protein ratio of 60:40 to 70:30 and most preferably a whey protein to casein protein ratio of 60:40.

The composition may comprise the WPC at a level of about 0.5 grams per litre (g/L) to about 10 g/L. Preferably, the WPC is present at a level of about 1 g/L to about 9 g/L. More preferably, the WPC is present in the composition at a level of about 3 g/L to about 8 g/L. Alternatively, the composition may comprise the WPC at a level of about 0.06 grams per 100 kilocalories (g/100 kcal) to about 1.5 g/100 kcal. Preferably, the WPC is present at a level of about 0.3 g/100 kcal to about 1.4 g/100 kcal. More preferably, the WPC is present in the composition at a level of about 0.4 g/100 kcal to about 1 g/100 kcal. Alternatively, the WPC is present in the composition at a level of about 6.5 wt % to about 12.0 wt % on a dry weight basis, preferably about 7.9 wt % to about 10.3 wt %.

The MFGM may be provided by an enriched milk product. The enriched milk product has a milk fat content in a range of between 14 to 20 wt %. The enriched milk product may be formed by fractionation of non-human milk, such as bovine milk. Alternatively, the enriched milk product is available commercially, including under the trade name Lacprodan MFGM-10, available from Arla Food Ingredients.

The enriched milk product may have a total protein level in a range of between 20% and 90%; preferably, the enriched milk product has a total protein level in a range of between 65% and 80%.

The enriched lipid fraction may be produced by any number of fractionation techniques. These techniques comprise, but are not limited, to melting point fractionation, organic solvent fractionation, super critical fluid fractionation, and any variants and/or any combination thereof.

The composition may comprise the enriched milk product at a level of about 0.5 grams per litre (g/L) to about 10 g/L. Preferably, the enriched milk product is present at a level of about 1 g/L to about 9 g/L. More preferably, the enriched milk product is present in the composition at a level of about 3 g/L to about 8 g/L. Alternatively, the composition may comprise the enriched milk product at a level of about 0.06 grams per 100 kilocalories (g/100 kcal) to about 1.5 g/100 kcal. Preferably, the enriched milk product is present at a level of about 0.3 g/100 kcal to about 1.4 g/100 kcal. More preferably, the enriched milk product is present in the composition at a level of about 0.4 g/100 kcal to about 1 g/100 kcal. Alternatively, the enriched milk product is present in the composition at a level of about 6.5 wt % to about 12.0 wt % on a dry weight basis, preferably about 7.9 wt % to about 10.3 wt %.

The MFGM may be provided by buttermilk. Buttermilk, in the context of the present disclosure, refers to an aqueous by-product of different milk fat manufacturing processes, especially the butter making process. Buttermilk is a concentrated source of MFGM components compared to other milk sources. Buttermilk includes dry buttermilk, which is defined as having a protein content of not less than 30%, and dry buttermilk product, which is defined as having a protein content of less than 30%. Both types of dry buttermilk have a minimum fat content of 4.5% and a moisture maximum of 5%. Cultured buttermilk is also within the contemplation of this disclosure. Buttermilk contains components such as lactose, minerals, oligosaccharides, immunoglobulins, milk lipids, and milk proteins, each of which is found in the aqueous phase during certain dairy cream processing steps.

Buttermilk may be obtained through different processes, such as: churning of cream during production of butter or cheese; production of variants of butter such as sweet cream butter, clarified butter, butterfat; production of anhydrous milk fat (butter oil) from cream or butter; removal of the fat-free dry matter and water from milk, cream, or butter, which is required to make anhydrous milk fat, yields buttermilk as a by-product; removal can be accomplished by mechanical- and/or chemical-induced separation; or, production of anhydrous milk fat (butter oil) from blending secondary skim and β-serum (and/or butter serum) streams together, respectively.

When the MFGM is provided by buttermilk, the composition may comprise buttermilk at a level of about 0.5 grams per litre (g/L) to about 70 g/L. Preferably, the buttermilk is present at a level of about 1 g/L to about 60 g/L. More preferably, buttermilk is present in the composition at a level of about 3 g/L to about 50 g/L. Alternatively, the composition may comprise buttermilk at a level of about 0.06 grams per 100 kilocalories (g/100 kcal) to about g/100 kcal. Preferably, the buttermilk is present at a level of about 0.3 g/100 kcal to about 8.5 g/100 kcal. More preferably, the buttermilk is present in the composition at a level of about 0.4 g/100 kcal to about 7 g/100 kcal.

It is hypothesised that administration of a synthetic nutritional composition comprising MFGM, during the time period and wherein MFGM is present in the specific amounts detailed in the description above, and defined in the claims, will be useful for improving neurological development in a paediatric subject from at least two years of age.

The MFGM may comprise phospholipids (such as phosphatidylethanolamine (PE), phosphatidylcholine (PC), phosphatidylserine (PS), and phosphatidylinositol (PI)), glycolipids, glycosphingolipids (such as sphingomyelin and gangliosides), other polar lipids, proteins, glycoproteins, triglycerides, cholesterol, 7-dehydrocholesterol, enzymes, or any combination thereof. Preferably, the MFGM provides at least one of phosphatidylcholine (PC), phosphatidylethanolamine (PE), phosphatidylserine (PS), phosphatidylinositol (PI), and/or derivatives thereof, and/or glycosphingolipids, and/or glycoproteins, and/or cholesterol.

More preferably, the MFGM provides at least one phospholipid selected from phosphatidylcholine (PC), phosphatidylethanolamine (PE), phosphatidylserine (PS), phosphatidylinositol (PI), and/or derivatives thereof; and/or sphingomyelin; and/or one or more gangliosides.

The amount of MFGM may be sufficient so as to provide at least 50 mg of phospholipids to the synthetic nutritional composition. The amount of MFGM may be sufficient so as to provide 50 mg to 30 g of phospholipids; preferably, 250 mg to 10 g of phospholipids. Alternatively, the synthetic nutritional composition comprises phospholipids in the range of about 50 mg/L to about 2000 mg/L, preferably about 100 mg/L to about 1000 mg/L, more preferably about 150 mg/L to about 550 mg/L of phospholipids. Alternatively, the synthetic nutritional composition comprises phospholipids in the range of about 6 mg/100 kcal to about 300 mg/100 kcal, preferably about 12 mg/100 kcal to about 150 mg/100 kcal, more preferably about 18 mg/100 kcal to about 85 mg/100 kcal.

The amount of MFGM may be sufficient so as to provide at least 10 mg of sphingomyelin to the synthetic nutritional composition. The amount of MFGM may be sufficient so as to provide 10 mg to 5 g of sphingomyelin; preferably, 50 mg to 1 g of sphingomyelin. Alternatively, the synthetic nutritional composition comprises sphingomyelin in a range of about 10 mg/L to about 200 mg/L, preferably about 30 mg/L to about 150 mg/L, more preferably about 50 mg/L to about 140 mg/L. Alternatively, the synthetic nutritional composition comprises sphingomyelin in a range of about 1 mg/100 kcal to about 30 mg/100 kcal, preferably about 3.5 mg/100 kcal to about 24 mg/100 kcal, more preferably about 6 mg/100 kcal to about 21 mg/100 kcal.

The amount of MFGM may be sufficient so as to provide at least 0.5 mg of gangliosides to the synthetic nutritional composition. The amount of MFGM may be sufficient so as to provide 0.5 mg to 3 g of gangliosides; preferably, 6 mg to 600 mg of gangliosides. Alternatively, the synthetic nutritional composition comprises gangliosides in a range of about 2 mg/L to about 40 mg/L, preferably about 6 mg/L to about 35 mg/L, more preferably about 9 mg/L to about 30 mg/L. Alternatively, the synthetic nutritional composition comprises gangliosides in a range of about 0.25 mg/100 kcal to about 6 mg/100 kcal, preferably about 0.7 mg/100 kcal to about 5.2 mg/100 kcal, more preferably about 1.1 mg/100 kcal to about 4.5 mg/100 kcal.

It is hypothesised that administration of a synthetic nutritional composition comprising the phospholipids content, sphingomyelin content and/or gangliosides content as detailed above, during the time period and wherein MFGM is present in the specific amounts detailed in the description above, and defined in the claims, will be useful for improving neurological development in a paediatric subject from at least two years of age.

The synthetic nutritional composition may further comprise lactoferrin.

Lactoferrin is a multifunctional iron-binding glycoprotein naturally present in milk. The lactoferrin may comprise human lactoferrin produced by a genetically modified organism, humanised lactoferrin, non-human lactoferrin, or a combination thereof. The non-human lactoferrin may comprise bovine lactoferrin, porcine lactoferrin, equine lactoferrin, buffalo lactoferrin, goat lactoferrin, murine lactoferrin, or camel lactoferrin.

Lactoferrin is known to have antimicrobial properties. Preclinical data reported in Mastromarino et al., Biometals 27, 1077-1086 (2014) suggest a potential modulatory role for lactoferrin on the gut microbiota. There is also emerging preclinical evidence for a potential role of lactoferrin in brain development and cognitive function. Preclinical studies in animal models have reported that lactoferrin can exert effects on learning, and memory (Chen Y et al, Mol Neurobiol 2015, 52(1):256-269).

The composition may comprise lactoferrin in the range of about 15 mg/100 kcal to about 300 mg/100 kcal. Preferably, the composition comprises lactoferrin in the range of about 60 mg to about 150 mg/100 kcal. More preferably, the composition comprises lactoferrin in the range of about 60 mg/100 kcal to about 100 mg/100 kcal.

When the composition comprises a reconstituted solution, the reconstituted solution may comprise lactoferrin in the range of about 0.5 mg/mL to about 1.5 mg/mL. Preferably, the reconstituted solution comprises lactoferrin in the range of about 0.6 mg/mL to about 1.3 mg/mL. Alternatively, the reconstituted solution may comprise lactoferrin in the range of about 0.1 grams per litre (g/L) to about 2 g/L. Preferably, the reconstituted solution comprises lactoferrin in the range of about 0.6 g/L to about 1.5 g/L.

The composition may comprise one or more prebiotics. The prebiotic may comprise oligosaccharides, polysaccharides, or any other prebiotics that comprise fructose, xylose, soya, galactose, glucose, mannose, or any combination thereof. More specifically, the prebiotic may comprise polydextrose (PDX), polydextrose powder, lactulose, lactosucrose, raffinose, glucooligosaccharides, inulin, fructooligosaccharides, isomaltooligosaccharides, soybean oligosaccharides, lactosucrose, xylooligosaccharides, chitooligosaccharides, mannooligosaccharides, aribino-oligosaccharides, sialyloligosaccharides, fucooligosaccharides, galactooligosaccharides (GOS), and gentiooligosaccharides.

The composition may comprise a prebiotic in the range of about 1.0 g/L to about 10.0 g/L of the composition. Preferably, the composition comprises a prebiotic in the range of about 2.0 g/L and about 8.0 g/L of the composition. Alternatively, the composition may comprise a prebiotic in the range of about 0.01 g/100 kcal to about 1.5 g/100 kcal. Preferably, the composition comprises a prebiotic in the range of about 0.15 g/100 kcal to about 1.5 g/100 kcal.

Preferably, the prebiotic comprises polydextrose (PDX) and/or galactooligosaccharides (GOS). The prebiotic may comprise at least 20% weight per weight (w/w) PDX, GOS, or a combination thereof.

The composition may comprise PDX in the range of about 1.0 g/L and 10.0 g/L. Preferably, the composition comprises PDX in the range of about 2.0 g/L and 8.0 g/L. Alternatively, the composition comprises PDX in the range of about 0.015 g/100 kcal to about 1.5 g/100 kcal. Preferably, the composition comprises PDX in the range of about 0.05 g/100 kcal to about 1.5 g/100 kcal. More preferably, the composition comprises PDX in the range of about 0.2 g/100 kcal to about 0.6 g/100 kcal.

The composition may comprise GOS in the range of about 0.015 g/100 kcal to about 1.0 g/100 kcal. Preferably, the composition comprises GOS in the range of about 0.2 g/100 kcal to about 0.5 g/100 kcal.

The composition may comprise PDX in combination with GOS. Advantageously, the combination of PDX and GOS may stimulate and/or enhance endogenous butyrate production by microbiota. The composition may comprise GOS and PDX in a total amount of at least about 0.015 g/100 kcal. The composition may comprise GOS and PDX in a total amount in the range of about 0.015 g/100 kcal to about 1.5 g/100 kcal. Preferably, the composition comprises GOS and PDX in a total amount in the range of about 0.1 g/100 kcal to about 1.0 g/100 kcal.

The composition may comprise one or more human milk oligosaccharides (HMOs). The HMO may comprise 2′-fucosyllactose (2FL), 3′-fucosyllactose (3FL), lacto-N-tetraose (LNT), lacto-N-neotetraose (LNnT), lacto-N-fucopentaose I (LNFP-I), 3′-sialyllactose (3SL), 6′-sialyllactose (6SL), or any combination thereof.

The composition may comprise an HMO in the range of about 0.01 g/L to about 5.0 g/L. Preferably, the composition comprises an HMO in the range of about 0.05 g/L to about 4.0 g/L of the composition. More preferably, the composition comprises an HMO in the range of about 0.05 g/L to about 2.0 g/L of the composition. Alternatively, the composition may comprise an HMO in the range of about 0.01 g/100 kcal to about 2.0 g/100 kcal. Preferably, the composition comprises an HMO in the range of about 0.01 g/100 kcal to about 1.5 g/100 kcal.

The composition may comprise a source of long-chain polyunsaturated fatty acids (LCPUFAs). The source of LCPUFAs may comprise docosahexaenoic acid (DHA), α-linoleic acid, γ-linoleic acid, linoleic acid, linolenic acid, eicosapentaenoic acid (EPA), arachidonic acid (ARA), or any combination thereof. Preferably, the composition comprises a source of LCPUFAs comprising DHA, ARA, or a combination thereof.

The composition may comprise an LCPUFA in an amount of at least about 5 mg/100 kcal. The composition may comprise an LCPUFA in the range of about 5 mg/100 kcal to about 100 mg/100 kcal. Preferably, the composition comprises an LCPUFA in the range of about 10 mg/100 kcal to about 50 mg/100 kcal.

The composition may comprise DHA in the range of about 5 mg/100 kcal to about 80 mg/100 kcal. Preferably, the composition comprises DHA in the range of about 10 mg/100 kcal to about 20 mg/100 kcal. More preferably, the composition comprises DHA in the range of about 15 mg/100 kcal to about 20 mg/100 kcal.

The composition may comprise ARA in the range of about 10 mg/100 kcal to about 100 mg/100 kcal of ARA. Preferably, the composition comprises ARA in the range of about mg/100 kcal to about 70 mg/100 kcal. More preferably, the composition comprises ARA in the range of about 20 mg/100 kcal to about 40 mg/100 kcal.

The composition may comprise both DHA and ARA. The weight ratio of ARA:DHA may be in the range of about 1:3 to about 9:1. Preferably, the weight ratio of ARA:DHA is in the range of about 1:2 to about 4:1. The composition may comprise oils containing DHA and/or ARA. If utilised, the source of DHA and/or ARA may be any source known in the art such as marine oil, fish oil, single cell oil, egg yolk lipid, or brain lipid. The DHA and ARA may be sourced from single cell oils, DHASCO® and ARASCO® from DSM Nutritional Products, or variations thereof. The DHA and ARA may be in a natural form, provided that the remainder of the LCPUFA source does not result in any substantial deleterious effect on the subject. Alternatively, the DHA and ARA may be used in refined form.

The synthetic nutritional composition comprises a protein source comprising MFGM, which is also a fat or lipid source. The synthetic nutritional composition may comprise a protein source and/or a fat or lipid source in addition to MFGM, a carbohydrate source, or any combination thereof. The synthetic nutritional composition may comprise one or more: probiotics; 8-glucan; sialic acid; suitable composition ingredient; or, any combination thereof.

The composition may comprise at least one protein source, in addition to MFGM, or lactoferrin when present. The protein source provides protein to the composition. The protein source may comprise intact protein, partially hydrolysed protein, extensively hydrolysed protein, small amino acid peptides, or any combination thereof. The protein source may be derived from any mammalian animal milk protein or plant protein, as well as their fractions, or any combination thereof. The protein source may comprise bovine milk, caprine milk, whey protein, casein protein, soy protein, rice protein, pea protein, peanut protein, egg protein, sesame protein, fish protein, wheat protein, hydrolysed protein, or any combination thereof. Bovine milk protein sources may comprise, but are not limited to, milk protein powders, milk protein concentrates, milk protein isolates, non-fat milk solids, non-fat milk, non-fat dry milk, whey protein, whey protein isolates, sweet whey, acid whey, casein, acid casein, caseinate (e.g. sodium caseinate, sodium calcium caseinate, calcium caseinate), or any combination thereof.

The composition may comprise the protein source in the range of about 1 g/100 kcal to about 7 g/100 kcal. Preferably, the composition comprises a protein source in the range of about 3.5 g/100 kcal to about 4.5 g/100 kcal. The protein source may comprise from about 40% to about 85% whey protein and from about 15% to about 60% casein.

As noted above, the protein source may comprise a source of intact protein. The composition may comprise intact protein in the range of about 1 g/100 kcal to about 3 g/100 kcal. Preferably, the composition comprises intact protein in the range of about 1 g/100 kcal to about 2.5 g/100 kcal. More preferably, the composition comprises intact protein in the range of about 1.3 g/100 kcal to about 2.1 g/100 kcal. The protein source may comprise a combination of intact protein and partially hydrolysed protein, wherein the partially hydrolysed protein may have a degree of hydrolysis of between about 4% and 10%.

As also noted above, the protein source of the composition may comprise partially hydrolysed protein, extensively hydrolysed protein, or a combination thereof. The hydrolysed proteins may be treated with enzymes to break down some or most of the proteins that cause adverse symptoms with the goal of reducing allergic reactions, intolerance, and sensitisation. The proteins may be hydrolysed by any method known in the art. The terms “protein hydrolysates” or “hydrolysed protein” are used interchangeably herein and refer to hydrolysed proteins, wherein the degree of hydrolysis may be from about 20% to about 80%, or from about 30% to about 80%, or even from about 40% to about 60%.

The composition may comprise at least one fat or lipid source in addition to MFGM, wherein the fat or lipid source provides fat and/or lipid to the composition. Suitable fat or lipid sources for the composition may be any known or used in the art. The fat or lipid source may be present in the composition in addition to another fat or lipid source, such as a LCPUFA. The fat or lipid source may comprise animal sources, such as milk fat, butter, butter fat, or egg yolk lipid; marine sources, such as fish oils, marine oils, or single cell oils; vegetable and plant oils, such as corn oil, canola oil, sunflower oil, soybean oil, palm olein oil, coconut oil, high oleic sunflower oil, evening primrose oil, rapeseed oil, olive oil, flaxseed (linseed) oil, cottonseed oil, high oleic safflower oil, palm stearin, palm kernel oil, or wheat germ oil; medium chain triglyceride oils; emulsions and esters of fatty acids; or any combination thereof.

The composition may comprise a fat or lipid source in the range of about 1 g/100 kcal to about 10 g/100 kcal. Preferably, the composition comprises a fat or lipid source in the range of about 2 g/100 kcal to about 7 g/100 kcal of a fat or lipid source. More preferably the composition comprises a fat or lipid source in the range of about 2.5 g/100 kcal to about 6 g/100 kcal. Most preferably, the composition comprises a fat or lipid source in the range of about 3 g/100 kcal to about 4 g/100 kcal.

The composition may comprise at least one carbohydrate source, wherein the carbohydrate source provides carbohydrate to the composition. The carbohydrate source may be present in the composition in addition to another carbohydrate source, such as one or more prebiotics. The carbohydrate source may comprise lactose, glucose, fructose, sucrose, starch, maltodextrin, maltose, fructooligosaccharides, corn syrup, high fructose corn syrup, dextrose, corn syrup solids, rice syrup solids, or any combination thereof. Moreover, hydrolysed, partially hydrolysed, and/or extensively hydrolysed carbohydrates may be desirable for inclusion in the composition due to their easy digestibility. More specifically, hydrolysed carbohydrates are less likely to contain allergenic epitopes. The composition may therefore comprise a carbohydrate source comprising hydrolysed or intact, naturally or chemically modified, starches sourced from corn, tapioca, rice, or potato, in waxy or non-waxy forms, such as hydrolysed corn starch.

The composition may comprise a carbohydrate source in the range of about 5 g/100 kcal to about 25 g/100 kcal. Preferably, the composition comprises a carbohydrate source in the range of about 6 g/100 kcal to about 22 g/100 kcal. More preferably, the composition comprises a carbohydrate source in the range of about 12 g/100 kcal to about 14 g/100 kcal.

The composition may comprise one or more probiotics. The probiotic may comprise any Bifidobacterium species, any Lactobacillus species, or a combination thereof. Preferably, the probiotic is Bifidobacterium adolescentis (ATCC number 15703), Bifidobacterium animalis subsp. lactis, Bifidobacterium breve, Bifidobacterium longum subsp. infantis (B. infantis), Lactobacillus acidophilus, Lactobacillus gasseri (ATCC number 33323), Lactobacillus reuteri (DSM number 17938), Lactobacillus rhamnosus GG (LGG; ATCC number 53103), or any combination thereof. More preferably, the probiotic is LGG, B. infantis, or a combination thereof.

The probiotic may be viable or non-viable. The probiotic incorporated into the composition may comprise both viable colony-forming units and non-viable probiotic cell-equivalents. The probiotic may be naturally-occurring, synthetic, or developed through the genetic manipulation of organisms, whether such source is now known or later developed.

The composition may comprise a viable probiotic in the range of about 1×10⁴ colony forming units per 100 kilocalories (CFU/100 kcal) to about 1.5×10¹² CFU/100 kcal. Preferably, the composition comprises a viable probiotic in the range of about 1×10⁶ CFU/100 kcal to about 1×10⁹ CFU/100 kcal. More preferably, the composition comprises a viable probiotic in the range of about 1×10⁷ CFU/100 kcal to about 1×10⁸ CFU/100 kcal.

The composition may comprise p-glucan. Preferably, the p-glucan comprises β-1,3-glucan. Preferably, the β-1,3-glucan comprises β-1,3;1,6-glucan. The composition may comprise p-glucan present in the range of about 0.010 grams to about 0.080 grams per 100 g of composition. Alternatively, the composition may comprise p-glucan in the range of about 3 mg/100 kcal to about 17 mg/100 kcal. Preferably, the composition comprises β-glucan in the range of about 4 mg/100 kcal to about 17 mg/100 kcal.

The composition may comprise sialic acid. Mammalian brain tissue contains the highest levels of sialic acid as sialic acid is incorporated into brain-specific proteins, such as the neural cell adhesion molecule (NCAM) and lipids (e.g. gangliosides). Sialic acid is therefore believed to play an important role in neural development and function, learning, cognition, and memory.

The composition may comprise sialic acid provided by an inherent source (such as an enriched milk product), exogenous sialic acid, sialic acid from sources (such as cGMP), or any combination thereof. The composition may comprise sialic acid in the range of about 100 mg/L to about 800 mg/L. Preferably, the composition comprises sialic acid in the range of about 120 mg/L to about 600 mg/L. More preferably, the composition comprises sialic acid in the range of about 140 mg/L to about 500 mg/L. Alternatively, the composition may comprise sialic acid in the range of about 1 mg/100 kcal to about 120 mg/100 kcal. Preferably, the composition comprises sialic acid in the range of about 14 mg/100 kcal to about 90 mg/100 kcal. More preferably, the composition comprises sialic acid in the range of about 15 mg/100 kcal to about 75 mg/100 kcal.

The synthetic nutritional composition may comprise one or more suitable composition ingredient, wherein the suitable composition ingredient comprises a vitamin, a mineral, choline, inositol, an emulsifier, a preservative, a stabiliser, or any combination thereof.

The composition may comprise all vitamins and minerals understood to be essential in the daily diet and in nutritionally significant amounts. Minimum requirements have been established for certain vitamins and minerals. Examples of minerals, vitamins and other nutrients optionally present in the nutritional compositions may include vitamin A, vitamin B1, vitamin B2, vitamin B6, vitamin B12, vitamin E, vitamin K, vitamin C, vitamin D, folic acid, inositol, niacin, biotin, pantothenic acid, choline, calcium, phosphorous, iodine, iron, magnesium, copper, zinc, manganese, chloride, potassium, sodium, selenium, chromium, molybdenum, taurine, and L-carnitine. Minerals are usually added in salt form. The presence and amounts of specific minerals and other vitamins will vary depending on the intended consumer whether it is a child or an infant.

As noted above, the composition may comprise choline. Choline is a nutrient that is essential for normal function of cells. Choline is a precursor for membrane phospholipids and it accelerates the synthesis and release of acetylcholine, a neurotransmitter involved in memory storage. Without wishing to be bound by theory, it is believed that dietary choline and docosahexaenoic acid (DHA) act synergistically to promote the biosynthesis of phosphatidylcholine and thus, help promote synaptogenesis in human subjects. Additionally, choline and DHA act synergistically to promote dendritic spine formation, which is important in the maintenance of established synaptic connections. The composition may comprise about 20 mg to about 100 mg of choline per 8 fl. oz. (236.6 mL) serving.

As noted above, the composition may comprise inositol. The inositol may be present as exogenous inositol, inherent inositol, or a combination thereof. The composition may comprise inositol in the range of about 10 mg/100 kcal to 40 mg/100 kcal. Preferably, the composition comprises inositol in the range of about 20 mg/100 kcal to 40 mg/100 kcal. Alternatively, the composition comprises inositol in the range of about 130 mg/L to about 300 mg/L.

The composition may comprise one or more emulsifier, as an emulsifier can increase the stability of the composition. The emulsifier may comprise, but is not limited to, egg lecithin, soy lecithin, alpha lactalbumin, monoglycerides, diglycerides, or any combination thereof.

The composition may comprise one or more preservative, as a preservative can extend the shelf-life of the composition. The preservative may comprise, but is not limited to, potassium sorbate, sodium sorbate, potassium benzoate, sodium benzoate, calcium disodium EDTA, or any combination thereof.

The composition may comprise one or more stabiliser, as a stabiliser can help preserve the structure of the composition. The stabiliser may comprise, but is not limited to, gum arabic, gum ghatti, gum karaya, gum tragacanth, agar, furcellaran, guar gum, gellan gum, locust bean gum, pectin, low methoxyl pectin, gelatine, microcrystalline cellulose, CMC (sodium carboxymethylcellulose), methylcellulose hydroxypropyl methyl cellulose, hydroxypropyl cellulose, DATEM (diacetyl tartaric acid esters of mono- and diglycerides), dextran, carrageenans, or any combination thereof.

The synthetic nutritional composition may be intended for a paediatric subject. The paediatric subject may be an infant or a child. The infant may be a vaginally-delivered infant. Alternatively, the infant may be an infant delivered by C-section.

The synthetic nutritional composition may comprise a nutritional supplement, a children's nutritional product, an infant formula, a human milk fortifier, a follow-up formula, a young child milk, or any other composition designed for an infant or a paediatric subject. The nutritional composition may be provided in any form known in the art. The nutritional composition may be provided in the form of a powder, a gel, a suspension, a paste, a solid, a liquid, a liquid concentrate, a reconstitutable powder, a reconstituted solution, or a ready-to-use product. The composition may be provided in an orally-ingestible form, wherein the orally-ingestible comprises a food, a beverage, a tablet, a capsule, or a powder.

Preferably, the synthetic nutritional composition is in the form of a reconstitutable powder, a reconstituted solution, or a ready-to-use product. Most preferably, the synthetic nutritional composition is provided in the form of a reconstitutable powder.

The synthetic nutritional composition may be expelled directly into a subject's intestinal tract. The synthetic nutritional composition may be expelled directly into the gut. The synthetic nutritional composition may be formulated to be consumed or administered enterally under the supervision of a physician.

The synthetic nutritional composition may be suitable for a number of dietary requirements. The composition may be kosher. The composition may be a non-genetically modified product. The composition may be sucrose-free. The composition may also be lactose-free. The composition may not contain any medium-chain triglyceride oil. No carrageenan may be present in the composition. The composition may be free of all gums.

The scope of the present invention is defined in the appended claims. It is to be understood that the skilled person may make amendments to the scope of the claims without departing from the scope of the present disclosure.

The foregoing detailed description has been provided by way of explanation and illustration, and is not intended to limit the scope of the appended claims. Many variations in the presently preferred embodiments illustrated herein will be apparent to one of ordinary skill in the art, and remain within the scope of the appended claims and their equivalents.

The present invention will now be described in relation to the following non-limiting Examples.

### EXPERIMENTAL SECTION

### Example 1—Follow-Up Study

The inventors then conducted a follow-up study to the study detailed in Li et al., J Pediatr 2019, 215, 24-31 on the participants who completed study feeding through day 365.

292 participants were eligible to take part in the follow-up study (Control: 148, MFGM+Lf: 144). 116 participants of between 5.5 to 6 years of age enrolled and completed follow-up assessments (Control: 59, MFGM+Lf: 57). Follow-up assessments were carried out as outlined below.

### Wechsler Preschool & Primary Scale of Intelligence

The Wechsler Preschool & Primary Scale of Intelligence (4th Edition; WPPSI-IV, adapted for the Chinese population) was carried out for each participant. The primary outcome was a standardized IQ test for ages 4 years 0 months to 6 years 11 months and secondary outcomes were Verbal Comprehension (VC), Visual Spatial (VS), Fluid Reasoning (FR), Working Memory (WM) and Processing Speed (PS). Composite scores for each variable were analysed separately by Analysis of Variance (ANOVA) to adjust for variables including study group, site, and sex and by Analysis of Covariance (ANCOVA) post-hoc to adjust for additional participant and family demographic variables (number of family members in the household, monthly average family income, mother and father highest education, years of early education completed prior to primary school and exposure to smoking in the home). The results are detailed in Table 1.

TABLE 1
WPPSI-IV Results

| | ANOVA | | | ANCOVA | | |
|---|---|---|---|---|---|---|
| Composite | | MFGM + | p- | | MFGM + | p- |
| Score | Control | Lf | value | Control | Lf | value |
| VC | 93.5 ± | 96.4 ± | 0.139 | 92.3 ± | 94.3 ± | 0.287 |
| | 1.4 | 1.4 | | 2.7 | 2.9 | |
| VS | 95.3 ± | 100.6 ± | 0.027 | 92.3 ± | 98.2 ± | 0.014 |
| | 1.7 | 1.7 | | 3.4 | 3.6 | |
| FR | 97.5 ± | 101.1 ± | 0.067 | 94.0 ± | 97.3 ± | 0.094 |
| | 1.4 | 1.4 | | 2.8 | 3.0 | |
| WM | 101.4 ± | 102.0 ± | 0.820 | 102.6 ± | 103.2 ± | 0.831 |
| | 1.7 | 1.7 | | 3.5 | 3.8 | |
| PS | 100.0 ± | 107.1 ± | <0.001 | 98.6 ± | 105.4 ± | <0.001 |
| | 1.4 | 1.4 | | 2.8 | 3.0 | |
| Full Scale | 93.5 ± | 98.7 ± | 0.012 | 90.9 ± | 95.6 ± | 0.020 |
| IQ | 1.5 | 1.4 | | 2.9 | 3.0 | |

The Full-Scale IQ, Visual Spatial, and Processing Speed composite scores from the WPPSI-IV were significantly higher for MFGM+Lf vs the Control group. Other composite scores were also higher for MFGM+Lf vs the Control group, but not significantly so.

### Stroop Task

The Stroop Task measures inhibitory control and rule learning. The task involved inhibiting congruous responses to simple stimuli and providing an incongruous response. Two forms of this task were completed: (i) banana=red, apple=yellow and (ii) sun=night, moon=day. Each participant carried out 16 trials and the outcome variable was the number of correct recitations. The scores were analysed by a repeated measures mixed-model including study group, site, sex, and task.

The Stroop Task scores (mean±SE) were significantly higher in the MFGM+Lf group compared to the Control group (15.6±0.4 vs 13.2±0.4; p<0.001).

Dimensional Change Card Sort (DCCS) task

The DCCS task measures rule learning and flexibility in cognitive reasoning in three sequential phases:
Pre-switch phase (6 trials): child sorts multidimensional stimuli (coloured shapes) based on colour;
Post-switch phase (6 trials): sort same stimuli based on shape; and
Border phase (12 trials): sort criterion is based on border presence (i.e., border present=colour, border absent=shape).

The outcome variable was the number of correct answers. Scores were analysed by ANOVA including study group, site, and sex. The highest phase passed was analysed by Cochran-Mantel-Haenszel stratified by site and sex.

On the DCCS Task, there were no differences on pre- or post-switch phase scores, but the MFGM+Lf group had higher scores (7.4±0.27) than Control (6.5±0.28) in the border phase (p=0.013).

Significant group differences were also detected in the highest phase passed (p=0.039); border was the highest phase passed for more children in the MFGM+Lf group (18, 32%) compared to the Control group (7, 12%), whereas passing both pre- and post-switch was the highest phase achieved for more children in the Control group (49, 83%) compared to the MFGM+Lf group (36, 63%).

### Child Behaviour Checklist

A child behaviour checklist was carried out, which is a parent report measure used to detect behavioural and emotional problems in children and adolescents. No difference was observed between the groups.

### Conclusions

The inventors found that feeding infants formula with MFGM and Lf through 12 months of age led to improved neurological outcomes (relative to the control group) later in life between 5.5 and 6 years of age. The results suggest that the addition of MFGM and Lf to infant formula has persistent effects in supporting neurological development during childhood. Similar results to those observed in the follow-up study of Example 1 are expected in the absence of lactoferrin.

### Example 2—Clinical Study

A double-blind, randomised, controlled trial was carried out at the Institute of Nutrition and Food Technology (INTA) at the University of Chile. The protocol and the informed consent forms were approved by the Institutional Review Board of INTA. Participants were randomised to an experimental formula (EF) and a control formula (SF). A reference group of infants receiving human milk (HM) was also enrolled.

Infants eligible for randomisation to study formula were consuming infant formula as the sole source of nutrition for at least 48 hours prior to randomisation.

Infants eligible for the Human Milk Reference group were consuming mother's own breast milk as the exclusive source of nutrition with the intent to feed mother's own breast milk through approximately 180 days of age.

Additional inclusion criteria for all infants were:
Singleton birth;
Up to 120 days of age at study registration or randomisation;
Birth weight between 2500 to 4500 g;
Gestational age between 37 and 42 weeks;
History of normal growth (weight between and inclusive of the 10th and 90th percentiles on the WHO growth chart [28]); and
parents agreed not to enroll the infant in another interventional study and signed informed consent.

The exclusion criteria included:
use of complementary feeding;
history of underlying endocrine, metabolic, or chronic diseases, congenital malformation, or any other condition that could interfere with the ability of the infant to ingest food, or to have normal growth and development;
evidence
of feeding difficulties or formula intolerance;
immunodeficiency; and
maternal illiteracy.

The experimental formula and the standard formula are shown below in Table 2.

TABLE 2
Study formula

| Nutrient | Unit | SF | EF |
|---|---|---|---|
| Protein | g | 1.9 | 1.9 |
| Fat | g | 5.3 | 5.3 |
| ARA | mg | 25 | 25 |
| DHA | mg | 17 | 17 |
| Carbohydrates | g | 11.4 | 11.4 |
| Vitamin A | IU | 300 | 300 |
| Vitamin D | IU | 60 | 60 |
| Vitamin E | IU | 2 | 2 |
| Vitamin K | μg | 9 | 9 |
| Thiamin | μg | 80 | 80 |
| Riboflavin | μg | 140 | 140 |
| Vitamin B6 | μg | 60 | 60 |
| Vitamin B12 | μg | 0.3 | 0.3 |
| Niacin | μg | 1000 | 1000 |
| Folic Acid | μg | 16 | 16 |
| Pantothenic Acid | μg | 500 | 500 |
| Biotin | μg | 3 | 3 |
| Vitamin C | mg | 12 | 12 |
| Choline | mg | 24 | 24 |
| Inositol | mg | 6 | 6 |
| Calcium | mg | 78 | 78 |
| Phosphorus | mg | 43 | 43 |
| Magnesium | mg | 8 | 8 |
| Iron | mg | 1.2 | 1.2 |
| Zinc | mg | 1 | 1 |
| Manganese | μg | 15 | 15 |
| Copper | μg | 75 | 75 |
| Iodine | μg | 15 | 15 |
| Selenium | μg | 2.8 | 2.8 |
| Sodium | mg | 27 | 27 |
| Potassium | mg | 108 | 108 |
| Chloride | mg | 63 | 63 |

The sources of protein for the SF were skimmed milk and whey protein concentrate (WPC). The sources of protein for the EF were skimmed milk, WPC and whey protein-lipid concentrate (5 g/L, source of bovine MFGM; Lacprodan MFGM-10, Arla Foods Ingredients).

The sources of fat for both formulas were a base blend of palm olein, soybean, coconut, and high oleic sunflower oils, fungal-derived single cell oil (source of ARA) and algal-derived single cell oil (source of DHA). ARA is arachidonic acid and DHA is docosahexaenoic acid.

The sources of carbohydrate for both formulas were available carbohydrates (source: lactose, galactose, glucose, fructose, epilactose): 10.8 g; and prebiotic oligosaccharides: 0.6 g (4 g/L; source: prebiotic blend of polydextrose [PDX] and galactooligosaccharides [GOS; 1:1 ratio])

All infants were enrolled before 120 days of age and infants receiving infant formula were randomised to receive a standard cow's milk-based infant formula (SF) or a similar formula with added whey protein-lipid concentrate (5 g/L MFGM-10; source of bovine MFGM) (EF) through 12 months of age to assess growth and tolerance.

A total of 347 infants were randomised (SF, n=174; EF, n=173) and the reference group of infants exclusively receiving human milk was also enrolled (HM, n=235).

### Sound Perception

In a subset of participants (SF, n=42; EF, n=35; HM, n=45), neurophysiologic syllable sound perception was evaluated at day 730 of life (2 years of age).

Auditory event-related potential (ERP) data were collected by carrying out electroencephalographic recordings in children using a geodesic sensor net (128 scalp sites; Electric Geodesic, Inc., Eugene, Oregon, USA).

The stimuli used were consonant—vowel syllables. The native familiar stimulus (NFS; also referred to as a standard stimulus) was a consonant-vowel (CV) syllable phonetically relevant in Spanish (ta). Two CV syllables were used as deviants: a native unfamiliar stimulus (NUS; also referred to as a native deviant) (da) and a foreign stimulus (FS; also referred to as a non-native deviant) (sha). Stimuli were presented in a mismatch negativity (MMN) paradigm that contained a NFS (80%), a NUS (10%) and an FS (10%) for a total of 1000 stimuli.

The amplitude and latency of the P1 wave response (first wave that appears after the stimulus onset) in a time period of between 100 and 250 ms was analysed.

In general, P1 amplitude was significantly less for NFS wave response compared to the NUS wave response (1.88±0.05 μV vs. 2.09±0.06 μV, p<0.004). P1 latency was also shorter for NFS compared to NUS (115.53±1.16 ms vs 124.25±0.89 ms, p<0.001). Overall, the inventors found that children at 24 months demonstrated differences in perception between familiar and unfamiliar auditory stimuli, the former being associated with lower ERP amplitude and shorter latency. This is to be expected for children at a young age. The differences in perception between familiar and unfamiliar auditory stimuli would be expected to decrease as language acquisition improves.

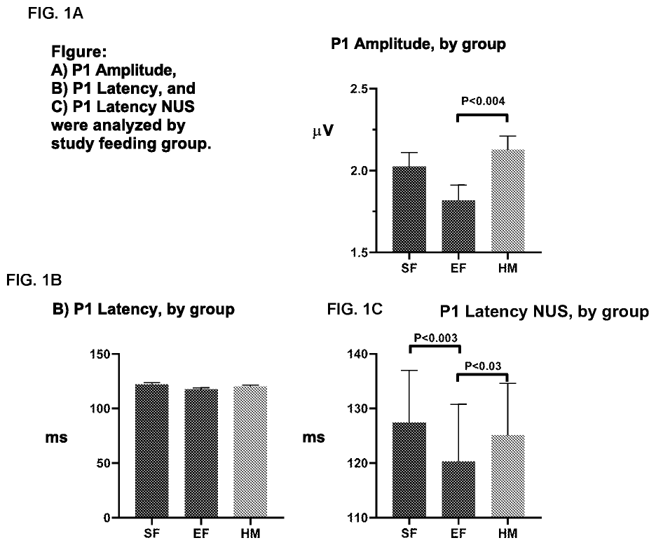

FIGS. 1A and 1B shows the amplitude and latency of the P1 wave response by feeding group. Overall P1 amplitude (native familiar stimuli, native unfamiliar stimuli and foreign stimuli combined) for the EF group was significantly lower compared to HM (1.81±0.09 μV vs 2.1±0.08 μV, p<0.04) (FIG. 1A). No significant differences in overall P1 latency were detected between feeding groups (FIG. 1B).

FIG. 1C shows the P1 latency for native unfamiliar stimuli (NUS). As shown in FIG. 1C, the response to NUS was significantly different by study feeding group. P1 latency was significantly shorter for the EF (120.29±10.4 ms) compared to HM and SF groups (125.1±9.5 ms, p<0.03 and 127.36±9.59 ms, p<0.003, respectively).

Overall, the inventors found that infants who received infant formula enriched with MFGM during the first year of life showed a lower ERP amplitude at two years of age compared to the standard formula-fed infants and breastfed infants, which may reflect a higher degree of maturation of neural circuits. The inventors also found that infants who received infant formula enriched with MFGM during the first year of life showed a lower ERP latency for native unfamiliar stimuli at two years of age, which may suggest improved myelination.

### SUMMARY

The results in Examples 1 and 2 suggest that the addition of MFGM in infant formulas supports neurological development in healthy infants and young children.

1. A synthetic nutritional composition comprising milk fat globule membrane (MFGM) in the range of about 10 μg/100 kcal to about 1500 mg/100 kcal for use in improving neurological development in a pediatric subject, wherein the synthetic nutritional composition is administered to the pediatric subject for at least three months during the first year of life, and wherein said administration of the synthetic nutritional composition improves neurological development of the pediatric subject from at least two years of age.

2. The synthetic nutritional composition according to claim 1, wherein the improvement in neurological development is an improvement in cognitive development.

3. The synthetic nutritional composition according to claim 2, wherein the improvement in cognitive development is selected from at least one of an improvement in the development of memory, verbal comprehension, visual spatial ability, fluid reasoning, mental processing speed, rule learning, executive function and language acquisition.

4. The synthetic nutritional composition for use according to claim 1, wherein the administration of the synthetic nutritional composition takes place for at least six months during the first year of life.

5. The synthetic nutritional composition according to claim 1, wherein the administration of the synthetic nutritional composition to the pediatric subject takes place from three months of age to six months of age.

6. The synthetic nutritional composition according to claim 1, wherein the improvement in the neurological development of the pediatric subject from at least two years of age persists until at least five years of age.

7. The synthetic nutritional composition according to claim 1, wherein the synthetic nutritional composition comprises MFGM in the range of about 15 μg/100 kcal to about 1000 mg/100 kcal.

8. The synthetic nutritional composition according to claim 1, wherein the synthetic nutritional composition comprises phospholipids in the range of about 6 mg/100 kcal to about 300 mg/100 kcal.

9. The synthetic nutritional composition according to claim 1, wherein the synthetic nutritional composition comprises sphingomyelin in a range of about 1 mg/100 kcal to about 30 mg/100 kcal.

10. The synthetic nutritional composition according to claim 1, wherein the synthetic nutritional composition comprises gangliosides in a range of about 0.25 mg/100 kcal to about 6 mg/100 kcal.

11. The synthetic nutritional composition according to claim 1, wherein the MFGM is provided by a whey protein concentrate.

12. The synthetic nutritional composition according to claim 11, wherein the synthetic nutritional composition comprises the whey protein concentrate in a range of about 6.5 to about 12.0 wt % on a dry weight basis.

13. The synthetic nutritional composition according to claim 1, wherein the MFGM is provided by an enriched milk product.

14. The synthetic nutritional composition according to claim 13, wherein the synthetic nutritional composition comprises the enriched milk product in the range of about g/100 kcal to about 1.5 g/100 kcal.

15. The synthetic nutritional composition for use according to claim 1, wherein the MFGM is provided by buttermilk.

16. The synthetic nutritional composition for use according to claim 15, wherein the synthetic nutritional composition comprises buttermilk in the range of about 0.06 g/100 kcal to about 10.5 g/100 kcal.

17. The synthetic nutritional composition according to claim 1, wherein the synthetic nutritional composition further comprises lactoferrin.

18. The synthetic nutritional composition according to claim 17, wherein the synthetic nutritional composition comprises lactoferrin in the range of about 5 mg/100 kcal to about 300 mg/100 kcal.

19. The synthetic nutritional composition according to claim 1, wherein the synthetic nutritional composition further comprises one or more of polydextrose (PDX), galactooligosaccharides (GOS), at least one human milk oligosaccharide (HMO), and a source of long-chain polyunsaturated fatty acids (LCPUFAs),
wherein the PDX, when present, is present in a range of about 0.015 g/100 kcal to about 1.5 g/100 kcal,
wherein the GOS, when present, is present in a range of about 0.015 g/100 kcal to about 1.0 g/100 kcal,
wherein the at least one HMO, when present, is selected from the group consisting of 2′-fucosyllactose (2FL), 3′-fucosyllactose (3FL), lacto-N-tetraose (LNT), lacto-N-neotetraose (LNnT), lacto-N-fucopentaose I (LNFP-1), 3′-sialyllactose (3SL), 6′-sialyllactose (6SL), and any combinations thereof, and is present in a range of about 0.01 g/100 kcal to about 2.0 g/100 kcal, and
wherein the source of LCPUFAs, when present, comprises docosahexaenoic acid (DHA), arachidonic acid (ARA), or a combination thereof and is present in a range of about 5 mg/100 kcal to about 100 mg/100 kcal.
